(19) United States
(12) Patent Application Publication
(10) Pub. No.: US 2008/0146963 A1
(43) Pub. Date: Jun. 19, 2008

(54) Biopsy needle guide

(76) Inventors: Kenneth M. Crocker, St. John's (CA); J. Scott Sloka, St. John's (CA); Jane Rendell, St. John's (CA)

Correspondence Address: EDWARD H. OLDHAM, 20 JAMESON DRIVE, DUNDAS, ON, L9H 5A2, omitted

(21) Appl. No.: 11/640,393

(22) Filed: Dec. 18, 2006

(51) Int. Cl. A61B 10/00

(52) U.S. Cl. 600/567

(57) ABSTRACT

A biopsy needle guide comprising two tapes fastened together so that the upper tape foils an arch with the other tape (base). The lower side of the base tape is coated with an adhesive material so that the guide may be applied to the surface of the skin of a patient.

Both tapes are provided with a series of spaced annular apertures which are arranged in a predetermined pattern, which are formed of a material which is visible on a computer generated tomological display.

### FIELD OF THE INVENTION

This invention relates to a guide to enable a radiologist or other physician to more accurately locate and sample tissue located some distance below the surface of the skin. Usually the patient has had a previous computed tomography (CT) or magnetic resonance imaging (MRI) scan of the body system of concern that has localized the lesion of interest. The radiologist is then asked to perform a percutaneous (through skin) biopsy of the tissue of interest, under CT guidance (or MRI), to ascertain the nature of the lesion. The biopsied tissue is then sent for pathological evaluation.

Alternatively, a lesion such as an abscess may require placement of a drainage catheter, or other non-vascular interventional procedure, in which case, accurate placement of a needle in the lesion is the first step in gaining access to the lesion.

Currently, the physician localizes the level of the lesion within the body using the CT scan and then inserts a biopsy needle knowing the approximate direction and depth below the surface of the skin where the suspected abnormality is located. However, there is no practical method currently available to easily and accurately determine the angle and distance to the lesion and guide the biopsy needle along the appropriate path.

### SUMMARY OF THE INVENTION

It is to improve the accuracy of guiding a biopsy needle that this invention is directed. It is assumed that a physician will be supplied with some sort of cross section radiographic imaging such as computed topography (CT) or magnetic resonance imaging (MRI). It is in this manner that a series of two dimensional images are produced for the physician's use in performing a biopsy procedure. The images will typically show a tissue abnormality or other lesion from which the physician performing the biopsy must obtain fluid or tissue samples.

To do so the operating radiologist or other physician will use the guide of this invention to assist in more accurately guiding the biopsy needle to the selected site.

The guide itself is a very simple device comprising two co-operating members; the first member is a somewhat flexible base strip and is capable of being affixed to the patient using its adhesive backing or it is applied to the human body using adhesive strips. The second member is longer than the first and forms an arch, the ends of which attach to the base strip, and the apex of the arch is above the center of the base strip. Each of these strips are provided with multiple ranks of holes or foramens, each of which are larger than the diameter of commonly used biopsy needles. The two component strips can be constructed separately for assembly, or as a single unit. The device can be constructed of a radioopaque material, such as copper or other metal with the ranks of holes being demarcated by striations or undulations built into the device, or, alternatively the device may be constructed of a material which is radiolucent, so that the device itself will be invisible on the radiological image produced, but the positions of the ranks of holes will be clearly demarcated on the image produced because the holes or foramens are surrounded by (or adjacent to) a radioopaque material such as metal wire. These demarcations exist to highlight the foramens so that they may be distinguished radiologically during the CT biopsy. It is noted that analogous methods may be used to demarcate the foramens from MRI imaging. The guide itself may be shipped in its assembled form or it may be assembled on site. It is important that the holes or foramens in the guide show up on a computer generated tomographical image.

Initially, the patient will be situated on the CT table and a series of axial images will be acquired for initial localization of the lesion. A suitable approximate biopsy pathway and orientation is selected by the physician, by examining the preliminary images. To use the guide, it is affixed to the patient undergoing biopsy at the level of the tissue to be sampled, as determined on the preliminary scan, and more axial images are acquired with the biopsy guide included on the acquired images. A laser guided axial plane device (standard equipment on most modern CT scanners) may be used to denote the plane of biopsy on the patient's body, and thereby line up the biopsy pathway through selected foramens on both members. The physician then inserts the biopsy needle through the selected foramens in the guide using the appropriate holes or foramens (selected from the CT scan) to achieve the desired entry angle. A final series of images may be obtained at this time for confirmation of biopsy path and depth to the lesion. The biopsy needle continues to be inserted into the patient's body to the predetermined depth along the predetermined path. Since the depth and approach angle to the lesion or suspect tissue has been measured from previous axial images and because the device was adhesively attached to the patient at a location corresponding to the convenient location described above, the insertion of the biopsy needle should be successful. An additional series of images may be obtained at this time to confirm that the needle tip is within the appropriate tissue, and a tissue sample is obtained following the usual method.

PERTINENT PRIOR ART

| U.S. Patents | | |
|---|---|---|
| 6,203,499 | Imling et al | Mar. 20, 2001 |
| 6,097,994 | Novab | Aug. 1, 2000 |
| 5,220,926 | Jones | Jun. 22, 1993 |
| 5,053,042 | Bidwell | Oct. 1, 1991 |
| Re 36,461 | Russell et al | Dec. 21, 1999 |

### BRIEF DESCRIPTION OF THE DRAWINGS

FIG. 1 shows a prior art guide used in this field.

FIG. 2 is a perspective view of the device of the invention.

FIG. 3 is a perspective view of the device of this invention being used with a laser scanner.

FIG. 4 shows an illustration of the device in use of image guided biopsy procedures.

### DESCRIPTION OF THE PREFERRED EMBODIMENT

FIG. 1 shows a prior art guide 2 which is in common use in the field today where it is used to assist an operating physician with more precision for the insertion of a biopsy needle. The FIG. 1 shows a single wire 6 mounted on backing 4. Note that this device describes a single locator wire, the use of multiple wires will generally be utilized in practical applications. This device will show up as a single or a series of multiple lines on a computer generated image depending on the number of wires 6 used. Adhesive pads 8 hold the wire 6 in place on the patient. The problem of this prior art device is that it fails to give the correct angle of insertion of a biopsy needle during a biopsy operation.

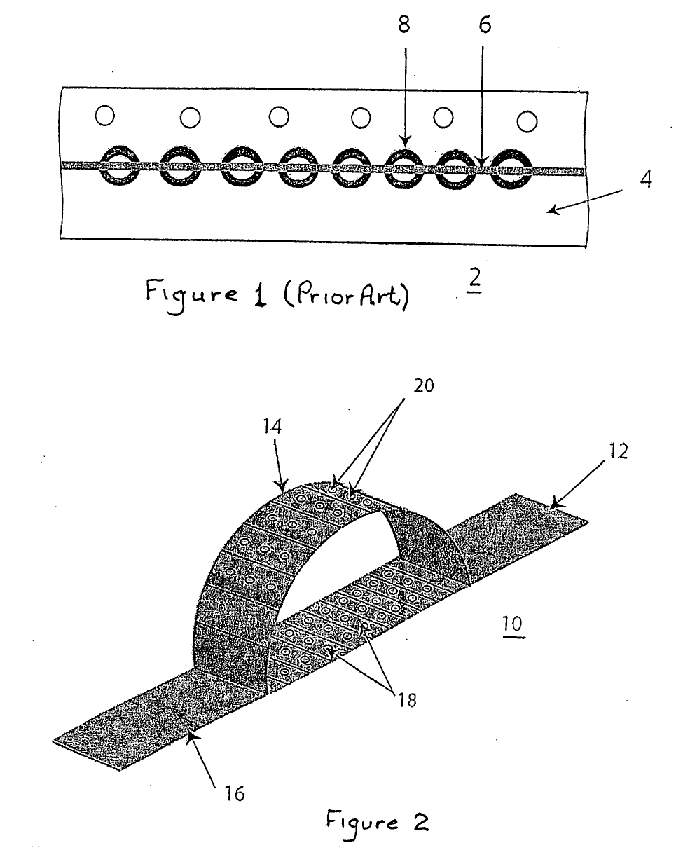

Referring now to FIG. 2, the device 10 of this invention is being shown comprising a pair of somewhat flexible radiolucent strips 12 and 14. Strip 12 which forms a base for the device 10 is a thin flexible flat strip having its lower surface 16 coated with a suitable adhesive material. Strip 12 is also provided with a series of circular holes or foramens 18 arranged in (in this instance) three parallel rows. The rows shown in FIG. 2 have been chosen to be three in number (this is an arbitrary decision) in this instance the rows are parallel and are equidistantly spaced. The spacing of the foramens or apertures 18 may be varied to suit the application and although the rows here number three, there may be more or less depending on the application.

Fastened to strip 12 is a second strip 14 in the form of an arch. Strip 14 is formed of the same material as base strip 12 and is fastened to strip 12 by any suitable means, such as gluing, stapling or staking to assure permanent fastening. Alternatively, the two components may be constructed as a single unit. Arch strip 14 also is provided with a series of foramens or apertures 20 which are of a similar diameter and shape as apertures 18. In any event the diameter of apertures or foramens 18 and 20 is slightly larger than the diameter of the biopsy needle being used. The strip 14 must be semi rigid or sufficiently stiff so as to maintain its profile under a variety of stressed conditions.

It is noted that strip 14 forms an arch with strip 12. The arch shape is not essential but the device 10 must have some separation between strips 12 and 14 in the working portion of the guide 10 to enable the operator to determine the angle of advance of the biopsy needle.

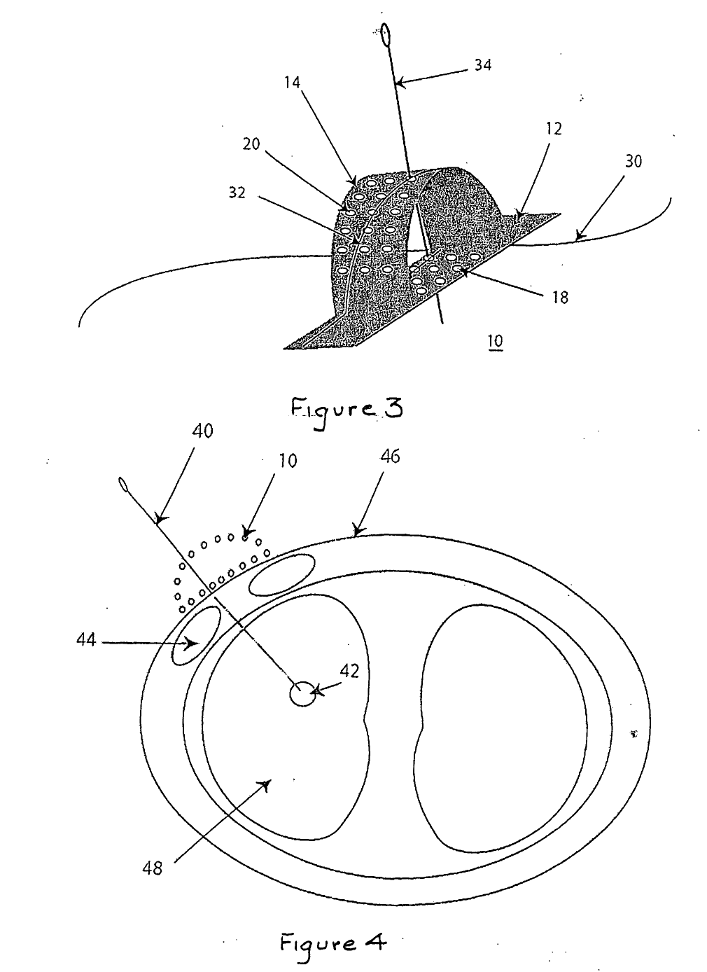

FIG. 3 shows the device 10 being used on the surface 30 of a patient in association with a laser trace 32 working with a CT scanner.

The laser trace 32 is generated by the CT scanner and the trace 32 is shown in the plane of the proposed biopsy. The intersection of the laser light with the apertures 18 and 20 provides the physician with the selection of which apertures must be employed in order that the lesion is intersected by the biopsy needle 34.

FIG. 4 is an illustration of how the device 10 may be used to guide a biopsy needle in a real life situation. Here needle 40 is shown intersecting the lesion 42. The device 10 is placed on the surface of the patient so that needle 40 will not intercept any of ribs 44, 46. Now needle 42 is angled by the holes in device 10 and directed to pass through the opening between ribs 44 and 46 and through lung 48 before intersecting lesion 42.

The device herein described is a radiolucent material having holes or foramens located therein for insertion of a biopsy needle. The number of holes or foramens in the rows of the two strips 14 and 16 must be fine enough to allow the radiologist ample opportunity to pick the “best” holes in strips 14 and 16 for obtaining the “best” entry angle in order to engage the biopsy needle with the lesion.

If a radioopaque device is utilized to assist the biopsy operation, it will be necessary to provide the radioopaque device with demarcations such as corrugations in which a series of holes or foramens are located. The device may be corrugated with the guide holes being located in the valleys of the corrugations. This feature allows the radiologist to select the proper angle for the insertion of a biopsy needle into the patient.

The advantages of this device are many:

The biopsy guide is a simple robust device which is easy to use in that it requires no significant training or familiarization in order to use the device.

The device is inexpesive, sterilizable and accurate. What's more, the guide may be made of radiolucent material so that it is minimally apparent on CAT scans. Alternatively, the device may be made solely of radioopaque material, reducing the cost of manufacturing. Alternatively the device may be made of analogous materials which are compatible with magnetic resonance imaging.

There are no moving parts to this device, so there are no adjustments requires in order to use this device.

The device will increase the accuracy and speed of biopsies and decrease side effects due to multiple biopsy attempts.

Many modifications and other embodiments of the invention will come to the mind of one skilled in the art having the benefit of the teachings presented in the foregoing descriptions and the associated drawings. Therefore, it is to be understood that the invention is not to be limited to the specific embodiments disclosed, and that the modifications and embodiments are intended to be included within the scope of the dependent claims.

What is claimed:

1. A biopsy needle guide comprising:
a base strip in the form of an elongated semi rigid tape having one side suitable for being coated with an adhesive material, said base strip having a plurality of foramens formed therein at first predetermined locations;
a second strip of tape of similar width as said base strip and being also in the form of a semi rigid tape; said second strip being secured at its ends to said base strip on a side opposite said one side in such a manner that said second strip forms an arch with said base strip;
said second strip also having a plurality of foramens formed therein at second predetermined locations.

2. A biopsy needle guide as claimed in claim 1 wherein said one side is coated with a suitable adhesive material.

3. A biopsy needle guide as claimed in claim 1 wherein said base strip is provided with a surface compatable for adhering to adhesive strips.

4. A biopsy needle guide as claimed in claim 2 in which said foramens in each strip are formed in at least three equidistantly spaced parallel rows.

5. A biopsy needle guide as claimed in claim 3 in which said foramens in each strip are formed in at least three equidistantly spaced parallel rows.

6. A biopsy needle guide as claimed in claim 4 wherein said foramens in said first and second strips are equidistantly spaced in said rows.

7. A biopsy needle guide as claimed in claim 5 wherein said foramens in said first and second strips are equidistantly spaced.

8. A biopsy needle guide as claimed in claims 2 or 3 wherein said strips are formed of a radiolucent material and said foramens in each strip are formed of a radioopaque material in the form of an annulus.

9. A biopsy needle guide as claimed in claims 2 or 3 wherein said strips are formed of a radiolucent material and said foramens in each strip are denoted with an encircling annulus of radioopaque material.

10. A biopsy needle guide as claimed in claims 2 or 3 wherein each foramen is of a circular shape having a circular opening of a size large enough to permit a biopsy needle to pass there through.

11. A biopsy needle guide comprising:
a base strip in the form of a semi rigid tape having one side suitable for being coated with an adhesive material, said base strip being formed of a radioopaque material which has a plurality of declivities or undulations formed therein at first predetermined locations,
a second strip of similar width as said base strip and also being in the form of semi rigid tape, said second strip being secured to said base strip on a side second strip forms an arch with said base strip,
said second strip also being formed of a radioopaque material and having a plurality of declivities or undulations formed therein at second predetermined locations.

12. A biopsy needle guide as claimed in claim 11 wherein said one side is coated with a suitable adhesive material.

13. A biopsy guide as claimed in claim 11 wherein said base strip is provided with a surface compatible for adhering to adhesive strips.

14. A method of guiding the angle of insertion of a biopsy needle into a patient to intercept a lesion comprising:
providing a computer generated tomographical image of said patient showing an anatomical slice of said patient which shows said lesion,
providing a guide for adhesively attaching to said patient;
and wherein said guide is formed of a base strip of a somewhat flexible material, having a second strip formed of a similar material to said base strip and having a similar width attached thereto at its ends;
said second strip being secured to said base in such a manner that it forms an arch with said base strip;
and wherein said base and second strips are each provided with a series of foramens designated by a radioopaque material located at predetermined locations thereon,
adhesively attaching said guide to said patient at a suitable location;
performing a computer generated tomographical image of said patient with said guide attached to said patient;
inserting a biopsy needle through selected foramens in said guide at such an angle as to intersect said lesion.
